(19) United States
(12) Patent Application Publication
(10) Pub. No.: US 2024/0424222 A1
(43) Pub. Date: Dec. 26, 2024

(54) A NEEDLESTICK INJURY MITIGATING SYRINGE SAFETY INJECTION GUN

(71) Applicant: COSMOAESTHETICS PTY LTD, Kew (AU)

(72) Inventor: Masoud JAFARZADEH, Kew (AU)

(21) Appl. No.: 18/699,535

(22) PCT Filed: Oct. 14, 2022
(86) PCT No.: PCT/AU2022/051231

§ 371 Date: Apr. 8, 2024

(30) Foreign Application Priority Data
Oct. 15, 2021 (AU) 2021903314

(51) Int. Cl. A61M 5/32

(52) U.S. Cl. CPC A61M 5/3232

(57) ABSTRACT

A safety injection gun has a pistol grip and a standoff at a terminus of a forestock thereof. A syringe carriage moveably is engaged along the forestock between a loaded position and a fired position. A shield is operative along the forestock and configurable in an open configuration and a closed configuration. The syringe can be safely loaded into the syringe carriage when the syringe carriage is safely in the loaded configuration. Thereafter, the shield may be closed, and the syringe carriage retracted wherein the needle is safely enclosed by the shield and the standoff, thereby reducing likelihood of needlestick injury.

### FIELD OF THE INVENTION

This invention relates generally to type of apparatus for administering syringe injections whilst mitigating against needlestick injury.

### BACKGROUND OF THE INVENTION

Needlestick injuries are a common occupational hazard in the hospital setting. According to the International Health Care Worker Safety Centre, approximately 295,000 hospital-based healthcare workers experience occupational percutaneous injuries annually.

More than 20 bloodborne pathogens can be transmitted from contaminated needles or sharps, including human immunodeficiency virus (HIV), hepatitis B virus (HBV), and hepatitis C virus (HCV). A quick and appropriate response to a needlestick injury can greatly decrease the risk of disease transmission following an occupational exposure to potentially infectious materials.

However, in USA alone, needle stick injuries are estimated to cost $188.5 million annually.

The present invention seeks to provide a way which will overcome or substantially ameliorate at least some of the deficiencies of the prior art, or to at least provide an alternative.

It is to be understood that, if any prior art information is referred to herein, such reference does not constitute an admission that the information forms part of the common general knowledge in the art, in Australia or any other country.

### SUMMARY OF THE DISCLOSURE

There is provided herein a handheld safety injection gun which comprises a pistol grip having a grip and a forestock.

The safety injection gun further comprises a standoff at a terminus of the forestock. The standoff defines a skin contacting face.

The safety injection gun comprises a syringe carriage moveably engaged along forestock between a loaded position and a fired position. The syringe carriage is closer to the standoff in the fired position as compared to when the syringe carriage is in the loaded position.

The syringe carriage may comprise a clamp configured to hold commercially available syringes. The clamp is configurable in an open configuration and a clamped configuration. In the clamped configuration, the clamp is configured to clamp against sides of a barrel of the syringe.

The safety injection gun further comprises a shield along the forestock. The shield is configurable in an open configuration and a closed configuration. The shield defines a passage for the syringe carriage therethrough along the forestock when the shield is in the closed configuration.

The safety injection gun further comprises a firing mechanism to urge the syringe carriage from the loaded position towards the fired position.

The safety injection gun further comprises a trigger mechanism interfacing the firing mechanism and the syringe carriage. The trigger mechanism comprises a trigger.

The trigger mechanism is operative to release the syringe carriage from the loaded position so that the syringe carriage travels to the fired position.

The safety injection gun is configured to be operated by loading a syringe into the syringe carriage when the syringe carriage is safely in the fired position.

When the syringe carriage is in the fired position, the firing mechanism is devoid of potential energy so that the syringe carriage cannot be accidentally fired.

The clamp may then be closed to the clamped configuration so that the clamp clamps against sides of the barrel of the syringe.

The shield may then be closed to the closed configuration.

The safety injection gun may then be loaded by retracting the syringe carriage along the forestock to the loaded position so that a tip of a needle of the syringe is safely shielded by the shield and the standoff, thereby significantly mitigating risk of needlestick injury.

The skin contacting face of the standoff can then be placed against the skin of a patient whereafter the trigger is pulled to release the trigger mechanism to release the firing mechanism to urge the syringe carriage towards the fired position so that the tip of the needle extends beyond the skin contacting face of the standoff to penetrate the skin.

In embodiments, the safety injection gun may have further safety mechanisms including wherein the syringe carriage is prevented from being retracted or the trigger mechanism is prevented from being fired when the shield is in the open configuration.

The shield may be designed to allow access to the syringe carriage so that the syringe carriage can be manually retracted to the loaded position when the shield is in the closed configuration.

The shield may visibly conceal the syringe to reduce needle phobia in some patients. In embodiments, the safety injection gun may have an oscillator to oscillate a frame of the standoff to reduce pain sensation.

Other aspects of the invention are also disclosed.

### BRIEF DESCRIPTION OF THE DRAWINGS

Notwithstanding any other forms which may fall within the scope of the present invention, preferred embodiments of the disclosure will now be described, by way of example only, with reference to the accompanying drawings in which:

FIG. 1 shows a rear perspective view of a safety injection gun;

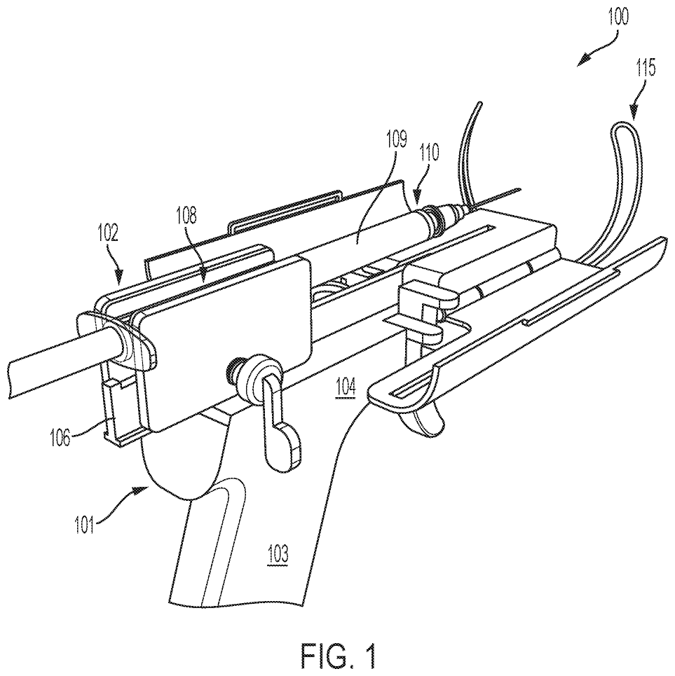

FIG. 2 shows a cross-sectional magnified perspective view of the gun;

FIG. 3 shows a side view of the gun;

FIG. 4 shows a front perspective view of the gun with a shield thereof open in accordance an embodiment; and

FIG. 5 shows a front perspective view of the gun with the shield closed.

### DESCRIPTION OF EMBODIMENTS

A safety injection gun 100 comprises a pistol grip 101 having a grip 103 and a forestock 104.

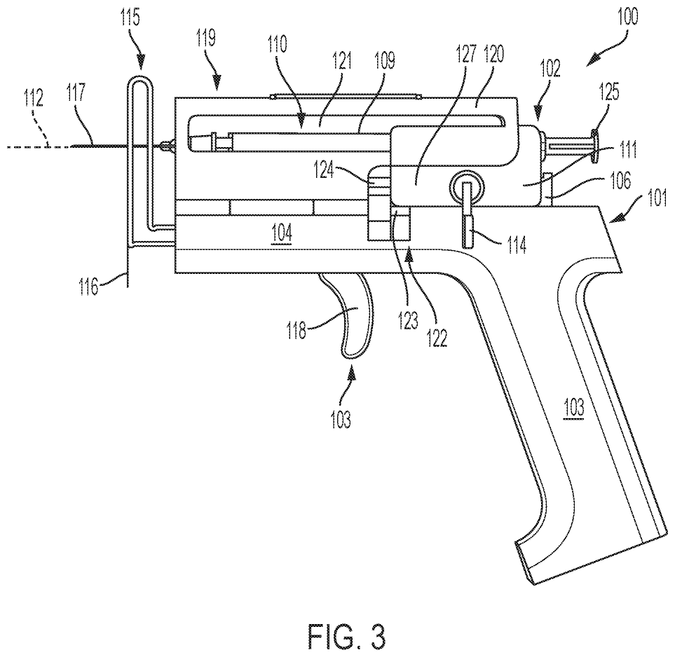

The safety injection gun 100 further comprises a standoff 115 at a terminus of the forestock 104. The standoff 115 defines a skin contacting face 116 as best shown in FIG. 3.

The safety injection gun 100 comprises a syringe carriage 102 moveably engaged along forestock 104 between a loaded position and a fired position. The syringe carriage 102 is closer to the standoff 115 in the fired position as compared to when the syringe carriage 102 is in the loaded position.

The syringe carriage 102 may comprise a clamp 108 configured to hold commercially available syringes 110. The clamp 108 is configurable in an open configuration and a clamped configuration. In the clamped configuration, the clamp 108 is configured to clamp against sides of a barrel 109 of the syringe 110.

The safety injection gun 100 further comprises a shield 119 along the forestock 104.

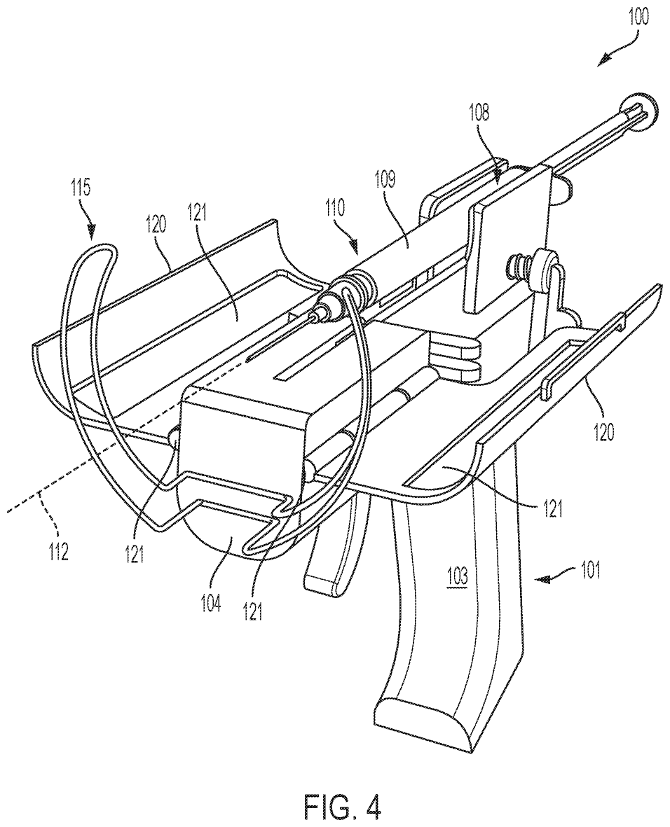

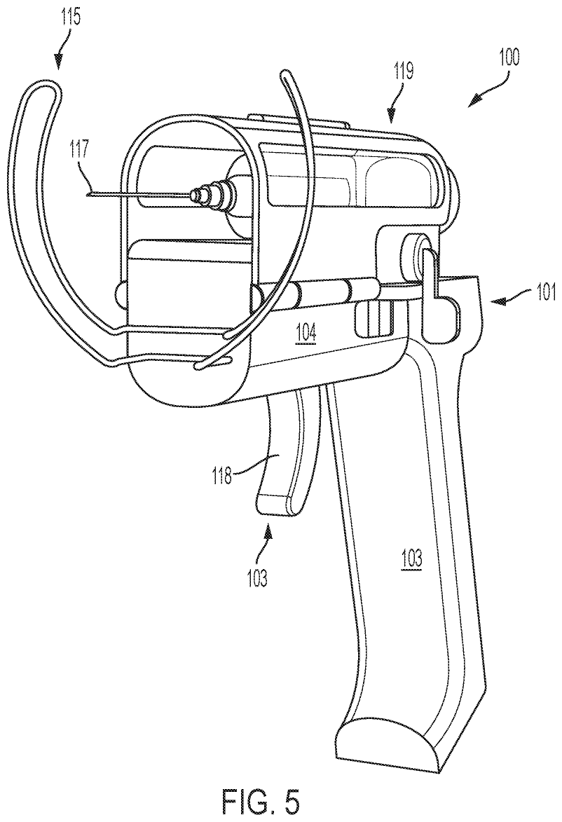

The shield 119 is configurable in an open configuration shown in FIG. 4 and a closed configuration shown in FIG. 5.

The shield 119 defines a passage for the syringe carriage 102 therethrough along the forestock 104.

The safety injection gun 100 further comprises a firing mechanism to urge the syringe carriage 102 from the loaded position towards the fired position.

The safety injection gun 100 further comprises a trigger mechanism 103 interfacing the firing mechanism and the syringe carriage 102. The trigger mechanism 103 comprises a trigger 118.

The trigger mechanism 103 is operative to release the syringe carriage 102 from the loaded position so that the syringe carriage 102 travels to the fired position.

The safety injection gun 100 is configured to be operated by loading the syringe 110 into the syringe carriage 102 when the syringe carriage 102 is in the fired position. When the syringe carriage 102 is in the fired position, the firing mechanism is without potential energy which prevents the syringe carriage 102 being fired accidentally when handling the syringe 110.

The clamp 102 of the syringe carriage 102 may be closed to the clamped configuration so that the clamp clamps against sides of the barrel 109 of the syringe 110.

The shield 119 may then be closed to the closed configuration.

The safety injection gun 100 may then be loaded by retracting the syringe carriage 102 along the forestock 104 to the loaded position so that a tip of a needle 117 of the syringe 110 is safely shielded by the shield and the standoff, thereby significantly mitigating risk of needlestick injury.

The skin contacting face 116 of the standoff 115 can then be placed against the skin of a patient whereafter the trigger 118 is pulled to release the trigger mechanism 103 to release the firing mechanism to urge the syringe carriage 102 towards the fired position so that the tip of the needle 117 extends beyond the skin contacting face 116 of the standoff 115 to penetrate the skin.

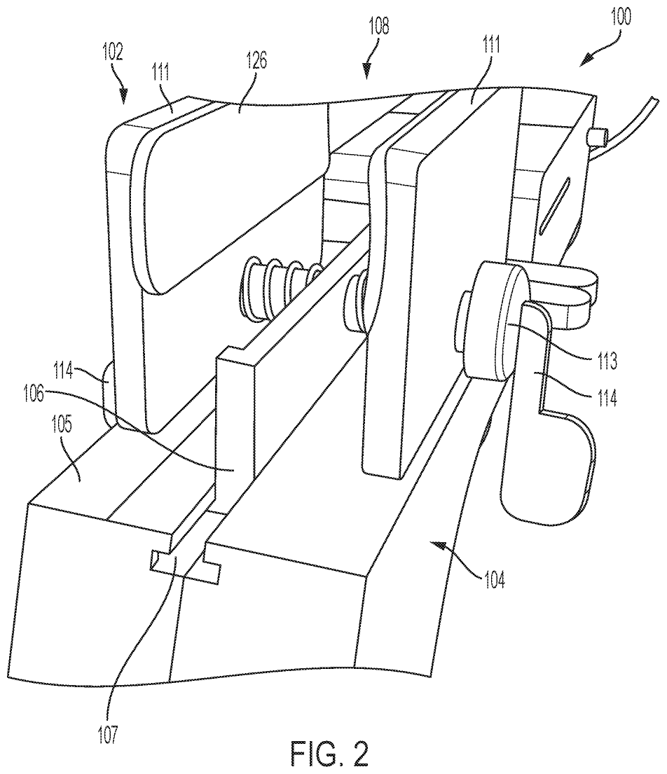

With reference to FIG. 2, the forestock 104 may define an upper surface 105 and wherein the carriage 102 slides across the upper surface 105.

The carriage 102 may comprise a profiled rail 106 slidably engaged within a channel 107 across the upper surface 105. In the embodiment shown, the profiled rail 106 is T-headed to fit within a conforming T-shaped channel 107 so that the rail 106 can slide along the channel 107 but cannot be pulled up out from the channel 107.

The clamp 108 may comprise opposing movable jaws 111 synchronously movable to keep the syringe 110 centred with respect to a syringe longitudinal axis 112. Each jaw 111 may comprise a shaped rubber friction pad 126 on an inside surface thereof.

The clamp 108 may comprise a screw 113 oppositely threaded for each movable jaw 111. The screw 113 may rotatably engaged through the rail 106. The screw may be turned by a turn handle 114. Turn handles 114 may be provided on both sides of the body 101 so that the clamp was a can be tightened with either hand. As will be apparent from the ensuing description, the turn handles 114 may be held individually or together between opposing fingers to retract the syringe carriage 102.

The standoff 115 may define the skin contacting face 116 as being substantially orthogonal with respect to the syringe longitudinal axis 112 shown in FIG. 3.

The safety injection gun 100 is configured so that a needle 117 tip of the syringe 110 is protected behind the skin contacting face 116 when the syringe carriage 102 is in the loaded position and protruding beyond of the skin contacting face 116 when the carriage 102 is in the fired position.

The standoff 115 may comprise a wire frame. The wire frame may be circular or semicircular in cross-section so as to partially or fully circumscribe the syringe longitudinal axis 112 as is evident from FIG. 4 so that, when pressed against skin, the skin forms a convexity coaxial with the needle tip 117. The wire frame may have an inner diameter of more than 5 cm.

The safety injection gun 100 may further comprise an oscillator causing the standoff 115 to oscillate. The oscillator may cause the wire frame to oscillate along the syringe longitudinal axis 112 to thereby gently oscillate the skin.

The safety injection gun 100 may comprise a battery power supply for the oscillator. The oscillator may comprise a piezoelectric or eccentric flywheel oscillator.

The trigger mechanism 103 may close a switch for a power supply circuit between the power supply and the oscillator. The trigger mechanism 103 may be configured to close the switch prior releasing the syringe carriage 102 from the loaded position.

For example, trigger mechanism 103 may be configured to close the switch when the trigger 118 is half pulled and only release the syringe carriage from the loaded position when the trigger 118 is fully pulled.

With reference to FIG. 4, the shield 119 may comprise a pair of side shield portions 120 pivotally engaged by hinges 121 either side of the forestock 104 and which close together to encase the syringe 110 therebetween in the manner shown in FIG. 5.

One or both portions 120 may comprise a viewing window 121 therealong.

The firing mechanism may comprise a tension spring concealed along the forestock 104 and operative between the syringe carriage 102 and the forestock 104. When the syringe carriage 102 is retracted, the tension spring is tensioned. When the trigger mechanism releases the syringe carriage 102, the tension spring releases potential energy to urge the syringe carriage 102 towards the fired position.

The shield 119 may allow the retraction of the carriage 102 when the shield 119 is in the closed configuration. With reference to FIG. 3, the side portions 120 may define longitudinal side openings 127 through which the syringe carriage 102 is accessible, such as by holding the turn handles 114 as alluded to above.

The portions 120 may be biased open and a catch 124 may hold the portions 120 in a closed configuration. The catch 124 may be released to open the portions 120.

The shield 119 may inhibit the trigger mechanism 103 so that the trigger mechanism 103 cannot be operated whilst the shield 119 is in the open configuration.

Furthermore, the shield 119 may inhibit the syringe carriage 102 so that the syringe carriage 102 cannot be retracted whilst the shield is open.

With reference to FIG. 3, the gun 100 may further comprise a travel limiter 122 configurable to limit the travel of the carriage 102 to one of short and long fired positions to control the insertion depth of the needle tip 117. In the long fired position, the syringe carriage 102 is closer to the standoff 115 as compared to when the syringe carriage 102 is in the short fired position.

The travel limiter 122 may comprise a selector 123 positionable in a catch position to catch the syringe carriage 102 at the short fired position and a non-catch position to allow the syringe carriage 102 to travel beyond the short fired position to the long fired position.

As is evident from FIG. 3, the selector 123 may be raised with respect to the upper surface 105 of the forestock 104 when in the catch position as compared to when in the non-catch position. As such, when the selector 123 is raised, the forward edge of the syringe carriage 102 strikes the selector 123, thereby preventing the syringe carriage 102 from travelling beyond the short fired position.

A method of injecting using the gun 100 may comprise opening the shield 119 and placing the barrel 109 of the syringe 110 placed between the movable jaws 111 of the clamp 108 when the syringe carriage 102 is in the fired position. The turn handles 114 are then turned to close the movable jaws 111 against the sides of the barrel 109 of the syringe 110.

In this way, the clamp 108 may engage syringe barrels 109 of typical commercially available syringes 110, including those having barrels 109 having outer diameters of 0.5 mm, 1 mm, 3 mm and 5 mm.

Thereafter, the shield 119 may be closed to the closed configuration with an underhand motion to push the shield portions 120 together. The shield 119 may prevent retracting of the syringe carriage 102 until the shield 119 is closed to the closed configuration.

The syringe carriage 102 may then be retracted to the loaded position. The side openings 127 allow the pulling back of the syringe carriage 102 while the shield is closed 119 by holding sides of the turn handles 114.

The position of the selector 123 of the travel limiter 122 may be selected to control the insertion depth of the needle tip 117. For example, the selector 123 may be raised for a shallower injection such as for a child and the selector 123 may be depressed for a deeper injection such as for an adult.

The gun 110 is then aimed at the injection site and gently pushed against the skin so that the wireframe of the standoff 114 forms the aforedescribed convexity facing the needle 117 tip.

The trigger 118 may then be half pulled, thereby causing the frame to gently isolate along the longitudinal axis 112 defined by the needle 117.

The trigger 118 may thereafter be fully pulled to cause the trigger mechanism 103 to release the syringe carriage 102 from the loaded position wherein the firing mechanism moves the syringe carriage 102 along the forestock 104 to the fired position so that the needle tip 117 penetrates the skin.

The plunger 125 of the syringe 113 may then be depressed to inject the medicine.

Thereafter, the gun 110 may be pulled away from the injection site, thereby pulling the needle tip 117 from the skin.

The shield release catch 124 may depressed to open the shield 119 and the grips 114 of the clamp 108 released to release the syringe 110 for discarding.

The foregoing description, for purposes of explanation, used specific nomenclature to provide a thorough understanding of the invention. However, it will be apparent to one skilled in the art that specific details are not required in order to practise the invention. Thus, the foregoing descriptions of specific embodiments of the invention are presented for purposes of illustration and description. They are not intended to be exhaustive or to limit the invention to the precise forms disclosed as obviously many modifications and variations are possible in view of the above teachings. The embodiments were chosen and described in order to best explain the principles of the invention and its practical applications, thereby enabling others skilled in the art to best utilize the invention and various embodiments with various modifications as are suited to the particular use contemplated. It is intended that the following claims and their equivalents define the scope of the invention.

1. A safety injection gun comprising:
a pistol grip having a grip and a forestock;
a standoff at a terminus of the forestock, the standoff defining a skin contacting face;
a syringe carriage moveably engaged along the forestock between a loaded position and a fired position, wherein the syringe carriage is closer to the standoff in the fired position as compared to when in the loaded position,
a shield operative along the forestock, the shield configurable in an open configuration and a closed configuration, the shield defining a passage for the syringe carriage therethrough along the forestock when the shield is in the closed configuration;
a firing mechanism to urge the syringe carriage from the loaded position towards the fired position;
a trigger mechanism operably interfacing the firing mechanism and the syringe carriage, the trigger mechanism operative to release the syringe carriage from the loaded position, wherein the safety injection gun is configured to be operated by:
placing a syringe into the syringe carriage when the syringe carriage is safely in the fired position;
closing the shield to the closed configuration;
loading the gun by retracting the syringe carriage along the forestock to the loaded position so that a tip of a needle of the syringe is safely shielded by the shield and the standoff;
placing the skin contacting face of the standoff against skin; and
pulling a trigger of the trigger mechanism to release the firing mechanism to urge the syringe carriage towards the fired position so that the tip of the needle extends beyond the skin contacting face of the standoff to penetrate the skin.

2. The safety injection gun as claimed in claim 1, wherein the syringe carriage comprises a clamp, the clamp configurable in an open configuration and a clamped configuration, wherein the clamp is configured to clamp against sides of a barrel of the syringe in the clamped configuration.

3. The safety injection gun as claimed in claim 1, wherein the firing mechanism comprises a tension spring along the forestock.

4. The safety injection gun as claimed in claim 2, wherein the clamp is defined by opposing moveable jaws synchronously movable with respect to maintain central alignment of the syringe with a syringe longitudinal axis.

5. The safety injection gun as claimed in claim 4, further comprising a screw which is oppositely threaded for each moveable jaw.

6. The safety injection gun as claimed in claim 5, further comprising turn handle for the screw.

7. The safety injection gun as claimed in claim 5, further comprising turn handles at both sides of the screw.

8. The safety injection gun as claimed in claim 1, wherein the standoff comprises a frame.

9. The safety injection gun as claimed in claim 8, wherein the frame is a wireframe.

10. The safety injection gun as claimed in claim 8, wherein the frame is at least one of semicircular and circular so as to at least one of partially circumscribe and circumscribe a syringe longitudinal axis.

11. The safety injection gun as claimed in claim 8, further comprising an oscillator causing the frame to oscillate with respect to the pistol grip.

12. The safety injection gun as claimed in claim 11, wherein the oscillator causes the standoff to oscillate along a syringe longitudinal axis with respect to the pistol grip.

13. The safety injection gun as claimed in claim 12, further comprising a power supply for the oscillator.

14. The safety injection gun as claimed in claim 13, wherein the trigger mechanism closes a switch of an electrical power supply circuit for the oscillator.

15. The safety injection gun as claimed in claim 13, wherein the trigger mechanism closes the switch prior releasing the syringe carriage from the loaded position when the trigger is pulled.

16. The safety injection gun as claimed in claim 1, wherein the shield comprises a pair of side shield portions pivotally engaged to sides of the forestock and which close together to encase the syringe therebetween.

17. The safety injection gun as claimed in claim 16, wherein the side shield portions are biased open.

18. The safety injection gun as claimed in claim 17, further comprising a catch holding the side shield portions in a closed configuration and wherein the catch is releasable to open the portions.

19. The safety injection gun as claimed in claim 1, wherein the shield inhibits the trigger mechanism so that the trigger mechanism cannot be triggered when the shield is in the open configuration.

20. The safety injection gun as claimed in claim 1, wherein the shield inhibits the syringe carriage so that the carriage cannot be retracted when the shield is in the open configuration.

21. The safety injection gun as claimed in claim 1, wherein the shield is shaped so that the syringe carriage can be accessed to be retracted when the syringe carriage is in the fired position and the shield is in the closed configuration.

22. The safety injection gun as claimed in claim 21, wherein the shield defines longitudinal side openings through which the carriage is accessible to be retracted along the forestock.

23. The safety injection gun as claimed in claim 1, further comprising a travel limiter configurable to limit the travel of the syringe carriage to one of a short fired position and a long fired position wherein the syringe carriage is closer to the standoff in the long fired position as compared to when the standoff is in the short fired position.

24. The safety injection gun as claimed in claim 23, wherein the travel limiter comprises a selector positionable in a catch position to limit the travel of the syringe carriage to the short fired position and a non-catch position to allow the syringe carriage to travel beyond the short fired position to the long fired position.

25. The safety injection gun as claimed in claim 24, wherein the selector is raised with respect to the forestock when in the catch position.
